(19) United States
(12) Patent Application Publication
(10) Pub. No.: US 2025/0313588 A1
(43) Pub. Date: Oct. 9, 2025

(54) NOVEL PYRROLOPYRIMIDINONE CARBOXAMIDE COMPOUND FOR INHIBITING CDK, STEREOISOMER THEREOF OR PHARMACEUTICALLY ACCEPTABLE SALT THEREOF, AND PHARMACEUTICAL COMPOSITION FOR TREATING CANCER, COMPRISING SAME AS ACTIVE INGREDIENT

(71) Applicants: Seoul National University R&DB Foundation, Seoul (KR); THE ASAN FOUNDATION, Seoul (KR); UNIVERSITY OF ULSAN FOUNDATION FOR INDUSTRY COOPERATION, Ulsan (KR)

(72) Inventors: Byeong Moon Kim, Seoul (KR); Seong Yun Jeong, Hanam-si, Gyeonggi-do (KR); Eun-Kyung Choi, Seoul (KR); Seok Soon Park, Bucheon-si, Gyeonggi-do (KR); Mi Ri Kwon, Seoul (KR); Ji Su Park, Gimhae-si, Gyeongsangnam-do (KR)

(21) Appl. No.: 18/865,281

(22) PCT Filed: May 10, 2023
(86) PCT No.: PCT/KR2023/006372

§ 371 Date: Nov. 12, 2024

(30) Foreign Application Priority Data
May 13, 2022 (KR) 10-2022-0059200

(51) Int. Cl. C07H 19/14; A61K 31/7064; A61P 35/00

(52) U.S. Cl. CPC C07H 19/14; A61K 31/7064; A61P 35/00

(57) ABSTRACT

The present invention relates to a pyrrolopyrimidinone carboxamide compound represented by Chemical Formula 1, a stereoisomer thereof or a pharmaceutically acceptable salt thereof, and a pharmaceutical composition for treating cancer, comprising the same as an active ingredient. Specifically, the compound increases plasma stability so that, until the compound reaches cancer cells, the structure thereof is maintained, and thus the compound exhibits anticancer effects greater than those of existing compounds.

### FIELD

The present invention relates to the synthesis and synthesis method of a novel CDK inhibitor that controls a cell cycle system in order to treat CDK-related cancer, and may be used in the development of therapeutic agents for intractable cancer.

### BACKGROUND ART

CDKs are serine/threonine protein kinases, which play a key role in regulating cell cycle and growth, and are attracting attention as targets for anticancer treatment due to their keen association with the growth and proliferation of cancer cells. In particular, unlike other CDK family members that generally play one role, CDK7, which plays various roles such as cell cycle regulation and RNA transcription, has become a target protein for various anticancer treatments, and research and development of inhibitors against it is being currently conducted in order to develop therapeutic agents for intractable cancers.

Although studies have been conducted mainly on covalent inhibitors such as THZ1 as CDK7 inhibitors, non-covalent inhibitors are currently under active development due to the problems of off-target effects and drug resistance.

Since compounds known in the related art have low plasma stability, experiments conducted on mice showed results that were less effective than expected, and to solve this problem, there is a need for developing a novel inhibitor that improves the plasma stability.

### DISCLOSURE

### Technical Tasks

Thus, the present inventors intended to improve the plasma stability by modifying the substituents of existing compounds.

The present invention is directed to providing a pyrrolopyrimidinone carboxamide compound represented by the following Chemical Formula 1, a stereoisomer thereof, or a pharmaceutically acceptable salt thereof:

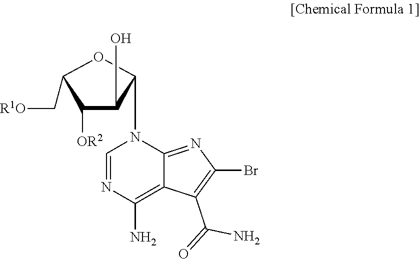

wherein R¹ is R³C(═O),
R³ is any one selected from the group consisting of a straight or branched C₃-C₁₀ alkyl, a C₃-C₁₀ cycloalkyl, and a C₆-C₁₀ aryl,
R² is hydrogen or R⁴C(═O), and R⁴ is any one selected from the group consisting of straight or branched C₃-C₆ alkyls, and when R³ is propyl or butyl, R² is not hydrogen.

Further, the present invention is directed to providing a pharmaceutical composition for preventing or treating cancer, including the pyrrolopyrimidinone carboxamide compound represented by Chemical Formula 1, a stereoisomer thereof, or a pharmaceutically acceptable salt thereof as an active ingredient.

In addition, the present invention is directed to providing a CDK inhibitor including the pyrrolopyrimidinone carboxamide compound represented by Chemical Formula 1.

Furthermore, the present invention is directed to providing a method for inhibiting CDK in a specimen or cell, the method including administering a pharmaceutically effective amount of the pyrrolopyrimidinone carboxamide compound represented by Chemical Formula 1 to the specimen.

Further, the present invention is directed to providing a method for preventing or treating cancer in a specimen, the method including administering a pharmaceutically effective amount of the pyrrolopyrimidinone carboxamide compound represented by Chemical Formula 1 to the specimen.

In addition, the present invention is directed to providing a use of the pyrrolopyrimidinone carboxamide compound represented by Chemical Formula 1 for preventing or treating cancer or tumor.

However, the technical problem to be achieved by the present invention is not limited to the aforementioned problems, and other problems that are not mentioned may be clearly understood by a person skilled in the art from the following description.

### Technical Solution

To achieve the above objects, the present inventors introduced a functional group having a larger volume into an ester group than the existing isopropyl structure that is easily decomposed, or introduced two functional groups into the compound.

One aspect of the present invention provides a pyrrolopyrimidinone carboxamide compound represented by the following Chemical Formula 1, a stereoisomer thereof, or a pharmaceutically acceptable salt thereof:

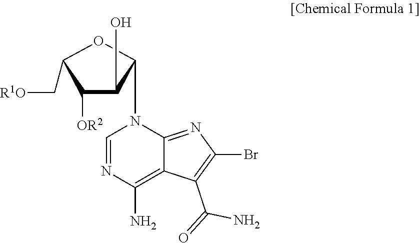

wherein R¹ is R³C(═O),
R³ is any one selected from the group consisting of a straight or branched C₃-C₁₀ alkyl, a C₃-C₁₀ cycloalkyl, and a C₆-C₁₀ aryl,
R² is hydrogen or R⁴C(═O), and R⁴ is any one selected from the group consisting of straight or branched C₃-C₆ alkyls, and
when R³ is propyl or butyl, R² is not hydrogen.

In an exemplary embodiment, R³ may be any one selected from the group consisting of isopropyl, butyl, pentyl, and adamantyl, and R⁴ may be any one selected from the group consisting of isopropyl, butyl, and pentyl.

In an exemplary embodiment, the compound represented by Chemical Formula 1 may be selected from the group consisting of
(1) (2S,3S,4S,5S)-5-(4-amino-6-bromo-5-carbamoyl-1H-pyrrolo[2,3-d]pyrimidin-1-yl)-4-hydroxy-2-((isobutyryloxy)methyl)tetrahydrofuran-3-yl isobutyrate;
(2) (2S,3S,4S,5S)-5-(4-amino-6-bromo-5-carbamoyl-1H-pyrrolo[2,3-d]pyrimidin-1-yl)-2-(((2,2-dimethylbutanoyl)oxy)methyl)-4-hydroxytetrahydrofuran-3-yl 2,2-dimethylbutanoate;
(3) ((2S,3S,4S,5S)-5-(4-amino-6-bromo-5-carbamoyl-1H-pyrrolo[2,3-d]pyrimidin-1-yl)-3,4-dihydroxytetrahydrofuran-2-yl)methyl 2,2-dimethylbutanoate;
(4) (2S,3S,4S,5S)-5-(4-amino-6-bromo-5-carbamoyl-1H-pyrrolo[2,3-d]pyrimidin-1-yl)-4-hydroxy-2-((pivaloyloxy)methyl)tetrahydrofuran-3-yl pivalate; and
(5) ((2S,3S,4S,5S)-5-(4-amino-6-bromo-5-carbamoyl-1H-pyrrolo[2,3-d]pyrimidin-1-yl)-3,4-dihydroxytetrahydrofuran-2-yl)methyl(3S,5S,7S)-adamantane-1-carboxylate.

Another aspect of the present invention provides a pharmaceutical composition for preventing or treating cancer, including the pyrrolopyrimidinone carboxamide compound represented by Chemical Formula 1, a stereoisomer thereof, or a pharmaceutically acceptable salt thereof as an active ingredient:

In an exemplary embodiment, the cancer may be liver cancer or lung cancer.

In an exemplary embodiment, the lung cancer may be non-small cell lung cancer.

In an exemplary embodiment, the composition may be characterized as having a CDK inhibitory action.

In an exemplary embodiment, the CDK may be any one selected from the group consisting of Cdk1, Cdk2, Cdk7, and Cdk9.

Still another aspect of the present invention provides a CDK inhibitor including the pyrrolopyrimidinone carboxamide compound represented by Chemical Formula 1.

Yet another aspect of the present invention provides a method for inhibiting CDK in a specimen or cell, the method including administering a pharmaceutically effective amount of the pyrrolopyrimidinone carboxamide compound represented by Chemical Formula 1 to the specimen.

Yet another aspect of the present invention provides a method for preventing or treating cancer in a specimen, the method including administering a pharmaceutically effective amount of the pyrrolopyrimidinone carboxamide compound represented by Chemical Formula 1 to the specimen.

Yet another aspect of the present invention provides a use of the pyrrolopyrimidinone carboxamide compound represented by Chemical Formula 1 for preventing or treating cancer or tumor.

### Advantageous Effects

The compound according to the present invention has increased plasma stability so that the structure thereof is maintained until the compound reaches cancer cells, and thus the compound exhibits a greater anticancer effect than existing compounds. By having a mechanism of action different from the majority of immune-based liver cancer therapeutic agents currently used, the compound according to the present invention can complement and replace immune anticancer agents through single or combined administration. In addition, it is possible to commercialize and enter the market in various aspects, such as using the compound according to the present invention as a therapeutic agent for cancer related to CDK7.

### DESCRIPTION OF DRAWINGS

FIG. 1 shows a method for synthesizing the compounds of the present invention.

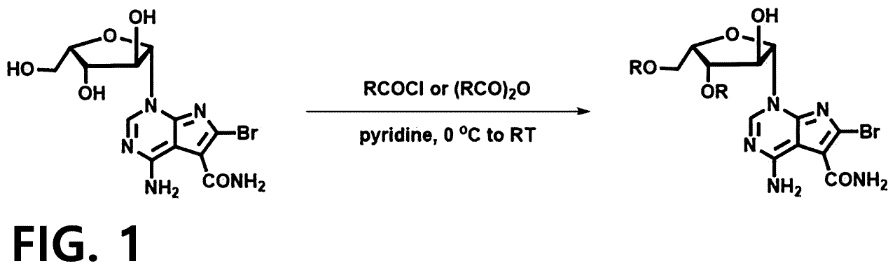

FIG. 2 shows compounds synthesized according to the present invention.

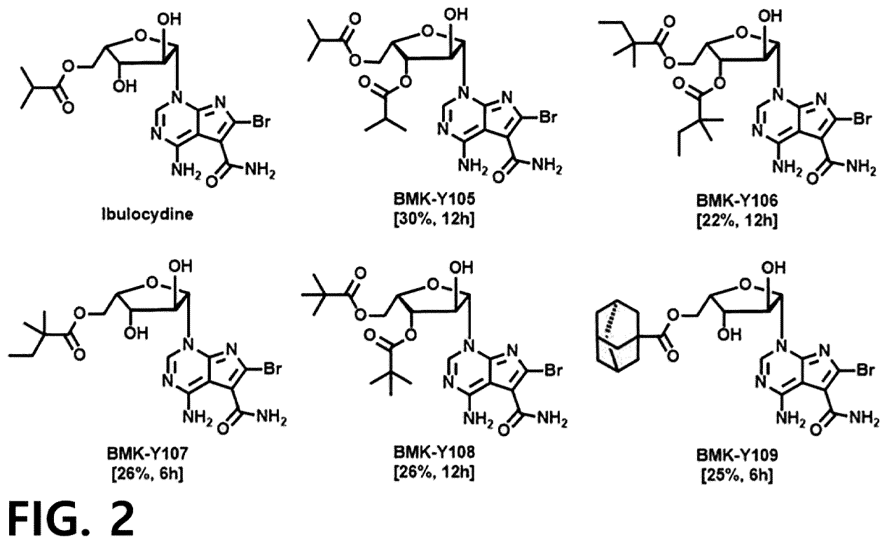

FIG. 3 shows the results of evaluating the anticancer efficacy of Y106 and Y108 in a mouse model transplanted with Huh-7 cells, a hepatocellular carcinoma cell line. A: Comparison of changes in cancer growth, B: Comparison of mouse body weight, C: Comparison of the size of the extracted cancer, D: Comparison of the weight of the extracted cancer.

FIG. 4 shows the results of evaluating the anticancer efficacy of Y106 and Y108 in a mouse model transplanted with Hep3B cells, a hepatocellular carcinoma cell line. A: Comparison of changes in cancer growth, B: Comparison of mouse body weight, C: Comparison of the size of the extracted cancer, D: Comparison of the weight of the extracted cancer.

FIG. 5 shows the results of evaluating the anticancer efficacy of Y106 and Y108 in a mouse model transplanted with A549 cells, a non-small cell lung cancer cell line. A: Comparison of changes in cancer growth, B: Comparison of mouse body weight, C: Comparison of the size of the extracted cancer, D: Comparison of the weight of the extracted cancer.

FIG. 6 shows the results of a cell colony formation assay performed on hepatocellular carcinoma cell lines Huh-7 and Hep3B cells.

### MODES OF THE INVENTION

The terms used in the present specification are used for descriptive purposes only and should not be construed as limiting. Singular expressions include plural expressions unless the context clearly indicates otherwise. In the present specification, it should be understood that the term “include” or “have” is intended to designate the presence of features, numbers, steps, operations, constituent elements, and parts described in the specification or combinations thereof, and does not exclude in advance the possibility of the presence or addition of one or more other features, numbers, steps, operations, constituent elements, and parts, or combinations thereof. Unless defined otherwise, all terms used herein, including technical or scientific terms, have the same meaning as commonly understood by a person with ordinary skill in the art to which exemplary embodiments pertain. Terms such as those defined in commonly used dictionaries should be interpreted as having a meaning consistent with the meaning in the context of the relevant art and should not be interpreted as ideal or overly formal in meaning unless explicitly defined in the present application.

Hereinafter, the present invention will be described in more detail.

As described above, the present inventors synthesized a novel compound, evaluated the anticancer efficacy of the novel compound in an animal model in which cancer tissue was formed, and confirmed that the novel compound had improved plasma stability and showed excellent effects compared to existing compounds.

Therefore, the present invention provides a pyrrolopyrimidinone carboxamide compound represented by the following Chemical Formula 1, a stereoisomer thereof, or a pharmaceutically acceptable salt thereof:

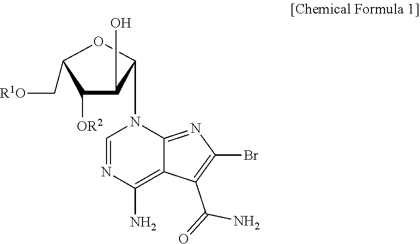

wherein R¹ is R³C(═O),
R³ is any one selected from the group consisting of a straight or branched C₃-C₁₀ alkyl, a C₃-C₁₀ cycloalkyl, and a C₆-C₁₀ aryl,
R² is hydrogen or R⁴C(═O), and R⁴ is any one selected from the group consisting of straight or branched C₃-C₆ alkyls, and
when R³ is propyl or butyl, R² is not hydrogen.

As used herein, “alkyl,” alone or as part of another substituent, means a fully saturated aliphatic hydrocarbon radical which is a straight or branched chain having the number of carbon atoms specified, unless otherwise specified. For example, “C₁-C₁₀ alkyl” refers to a straight or branched hydrocarbon radical containing 1 to 10 carbon atoms derived by removing one hydrogen atom from a single carbon atom of a parent alkane. In the context of the present invention, the term “alkyl” means “a C₁-C₁₀ alkyl,” preferably “a C₃-C₁₀ alkyl,” unless otherwise specified.

“Alkenyl” alone or as part of another substituent means a straight or branched chain which may be mono-or polyunsaturated, having the number of carbon atoms specified. For example, “C₂-C₈ alkenyl” means an alkenyl radical having 2, 3, 4, 5, 6, 7 or 8 atoms derived by removing one hydrogen atom from a single carbon atom of a parent alkane. In the context of the present invention, the term “alkenyl” means “a C₂-C₁₀ alkenyl,” preferably “a C₂-C₅ alkenyl,” unless otherwise specified.

“Alkynyl” alone or as part of another substituent means a straight or branched hydrocarbon radical which may be mono-or polyunsaturated, having the number of carbon atoms specified. For example, “C₂-C₈ alkynyl” means an alkynyl radical having 2 to 8 atoms derived by removing one hydrogen atom from a single carbon atom of a parent alkane. In the context of the present invention, the term “alkynyl” means “a C₂-C₁₀ alkynyl,” preferably “a C₂-C₅ alkynyl,” unless otherwise specified.

“Cycloalkyl” alone or as part of another substituent refers to a cyclic form of “alkyl,” “alkenyl,” and “alkynyl,” in which all ring atoms are carbon, unless otherwise stated. “Cycloalkyl” or “carbocycle” refers to a monocyclic group or a polycyclic group. When used in connection with a cycloalkyl substituent, the term “polycyclic” refers to both fused and non-fused alkyl ring structures. The “cycloalkyl” or “carbocycle” may form a bridged ring or spiro ring. A cycloalkyl group may have one or more double or triple bonds. The term “cycloalkenyl” refers to a cycloalkyl group having one or more sites of alkenyl unsaturation between the ring vertices. The term “cycloalkynyl” refers to a cycloalkyl group having one or more sites of alkynyl unsaturation between the ring vertices. When “cycloalkyl” is used in combination with “alkyl”, such as in C₃₋₈ cycloalkyl C₃₋₈ alkylene, the cycloalkyl moiety is meant to have the specified number of carbon atoms (for example, 3 to 8 carbon atoms) and the alkylene moiety has 1 to 8 carbon atoms. A typical cycloalkyl substituent has 3 to 8 ring atoms. Examples of cycloalkyl include cyclopentyl, cyclohexyl, 1-cyclohexenyl, 3-cyclohexenyl, cycloheptyl, and the like.

“Aryl” alone or as part of another substituent refers to a polyunsaturated aromatic hydrocarbon group containing 6 to 14 carbon atoms, may be a single ring or multiple rings (up to three rings), which may be fused or covalently linked together. Therefore, the phrase includes, but is not limited to, for example, a group such as phenyl, biphenyl, anthracenyl, and naphthyl. Non-limiting examples of an unsubstituted aryl group include phenyl, 1-naphthyl, 2-naphthyl, and 4-biphenyl. In the context of the present invention, the term “aryl” means “a C₆-C₁₂ aryl,” preferably “a C₆-C₁₀ aryl,” unless otherwise specified.

“Arylalkyl” or “aralkyl” refers to a monovalent alkyl group substituted with an aryl. Examples of arylalkyl include, but are not limited to, benzyl. In certain embodiments, both alkyl and aryl may be optionally substituted with one or more substituents as described herein. An example of arylalkyl is benzyl. “Alkoxy” refers to —ORd, wherein Rd is alkyl defined herein. Representative examples of an alkoxy group include methoxy, ethoxy, t-butoxy, trifluoromethoxy, and the like. In the context of the present invention, the term “alkoxy” means “a C₁-C₁₀ alkoxy,” preferably “a C₁-C₅ alkoxy,” unless otherwise specified.

“Alkoxyalkyl” refers to a monovalent alkyl group substituted with an alkoxy. For example, “C₁-C₅ alkoxy C₁-C₅ alkyl” means an alkyl radical having 1 to 5 carbon atoms derived by substituting one hydrogen atom with a C₁-C₅ alkoxy. Examples of alkoxyalkyl include, but are not limited to, methoxymethyl, ethoxymethyl, ethoxyethyl, and 2-methoxyethyl.

“Aryloxyalkyl” or “arylalkyloxyalkyl” refers to a monovalent alkyl group substituted with an aryloxy or arylalkyloxy. For example, “C₆-C₁₀ arylalkyloxy C₁-C₅ alkyl” means an alkyl radical having 1 to 5 carbon atoms derived by substituting one hydrogen atom with a C₆-C₁₀ arylalkyloxy.

“Alkoxyalkoxyalkyl” refers to a divalent alkyl group substituted by an alkoxy group. For example, “C₁-C₅ alkoxy(C₁-C₅ alkoxy)C₁-C₅ alkyl” means an alkyl radical having 1 to 5 carbon atoms derived by substituting two hydrogen atoms each with a C₁-C₅ alkoxy group.

“Acyl” refers to a —C(═O)RC group, where RC is alkyl, alkenyl, alkynyl, cycloalkyl, cycloalkenyl, aryl, heteroaryl, or heterocyclyl. Acyl includes —C(═O)CH₃ which is an “acetyl” group.

“Carbonyl” refers to a divalent —C(═O)— group. “Carboxy” or “carboxyl” refers to a —CO₂H group. “Carboxyl ester” or “carboxy ester” refers to a —C(═O)ORC group, where Rc is alkyl, alkenyl, alkynyl, cycloalkyl, cycloalkenyl, aryl, heteroaryl, or heterocyclyl.

“Alkoxycarbonyl” refers to —C(═O)ORd, where Rd is alkyl.

As used herein, each term (for example, “alkyl,” “heteroalkyl,” “aryl” and “heteroaryl”) includes both “unsubstituted” and optionally “substituted” forms of a specified radical, unless otherwise specified. Typically, each radical is substituted with 0, 1, 2, 3 or 5 substituents, unless otherwise specified. Examples of substituents for each type of radical are provided below.

“Substituted” refers to the substitution of one or more bonds to a carbon(s) or hydrogen(s) with a bond to non-hydrogen and non-carbon atom “substituents”, for example, the substituent is a halogen atom such as F, Cl, Br and I; an oxygen atom in a group such as a hydroxyl group, an alkoxy group, an aryloxy group and an acyloxy group; a sulfur atom in a group such as a thiol group, an alkyl and aryl sulfide group, a sulfone group, a sulfonyl group, and a sulfoxide group; a nitrogen atom in a group such as amino, alkylamine, dialkylamine, arylamine, alkylarylamine, diarylamine, alkoxyamino, hydroxyamino, acylamino, sulfonylamino, N-oxide, imide, and enamine; and other heteroatoms in several other groups, but is not limited thereto. Further, “substituent” includes a group in which one or more bonds to a carbon or hydrogen atom(s) are replaced by a higher order bond (for example, a double or triple bond) to a heteroatom, and for example, the heteroatom is oxygen in oxo, acyl, amido, alkoxycarbonyl, aminocarbonyl, carboxyl, and ester groups; nitrogen in a group such as imine, oxime, hydrazone, and nitrile. “Substituent” also include a group in which one or more bonds to a carbon or hydrogen atom(s) are replaced with a bond to a cycloalkyl, heterocyclic, aryl, and heteroaryl group. In particular, a representative “substituent” includes a group in which one or more bonds to a carbon or hydrogen atom are substituted with one or more bonds to a fluoro, chloro, or bromo group. Another representative “substituent” is another group containing a trifluoromethyl group and a trifluoromethyl group. Still another representative “substituent” includes those in which one or more bonds to a carbon or hydrogen atom are replaced with a bond to an oxygen atom, and the substituted alkyl group includes a hydroxyl group, an alkoxy group, or an aryloxy group. Yet another representative “substituent” is an alkyl group having amine, or a substituted or unsubstituted alkylamine, dialkylamine, arylamine, (alkyl)(aryl) amine, diary lamine, heterocyclylamine, diheterocyclylamine, (alkyl)(heterocyclyl) amine or (aryl)(heterocyclyl) amine. Yet another representative “substituent” includes those in which one or more bonds to a carbon or hydrogen atom(s) are replaced with a bond to an alkyl, cycloalkyl, aryl, heteroaryl, or heterocyclyl group. Groups defined herein may include prefixes and/or suffixes commonly used in the art to create additional well-recognized substituents.

In an exemplary embodiment, as R³, any one selected from the group consisting of a straight or branched C₃-C₁₀ alkyl, a C₃-C₁₀ cycloalkyl, and a C₆-C₁₀ aryl can be used without limitation, but R³ may be preferably any one selected from the group consisting of isopropyl, butyl, pentyl, and adamantyl. In addition, R⁴ may be any one selected from the group consisting of isopropyl, butyl, and pentyl.

As shown in Example 1, in an exemplary embodiment, the compound represented by Chemical Formula 1 may be selected from the group consisting of
(1) (2S,3S,4S,5S)-5-(4-amino-6-bromo-5-carbamoyl-1H-pyrrolo[2,3-d]pyrimidin-1-yl)-4-hydroxy-2-((isobutyryloxy)methyl)tetrahydrofuran-3-yl isobutyrate;
(2) (2S,3S,4S,5S)-5-(4-amino-6-bromo-5-carbamoyl-1H-pyrrolo[2,3-d]pyrimidin-1-yl)-2-(((2,2-dimethylbutanoyl)oxy)methyl)-4-hydroxytetrahydrofuran-3-yl 2,2-dimethylbutanoate;
(3) ((2S,3S,4S,5S)-5-(4-amino-6-bromo-5-carbamoyl-1H-pyrrolo[2,3-d]pyrimidin-1-yl)-3,4-dihydroxytetrahydrofuran-2-yl)methyl 2,2-dimethylbutanoate;
(4) (2S,3S,4S,5S)-5-(4-amino-6-bromo-5-carbamoyl-1H-pyrrolo[2,3-d]pyrimidin-1-yl)-4-hydroxy-2-((pivaloyloxy)methyl)tetrahydrofuran-3-yl pivalate; and
(5) ((2S,3S,4S,5S)-5-(4-amino-6-bromo-5-carbamoyl-1H-pyrrolo[2,3-d]pyrimidin-1-yl)-3,4-dihydroxytetrahydrofuran-2-yl)methyl(3S,5S,7S)-adamantane-1-carboxylate.

Furthermore, the present invention provides a pharmaceutical composition for preventing or treating cancer, including the pyrrolopyrimidinone carboxamide compound represented by Chemical Formula 1, a stereoisomer thereof, or a pharmaceutically acceptable salt thereof as an active ingredient:

As used herein, the term “pharmaceutical composition” refers to a mixture including a chimeric compound of the present invention and a pharmaceutically acceptable excipient such as a diluent or a carrier. The pharmaceutical composition includes a cosmetic composition as well as a composition for therapeutic use. According to some exemplary embodiments, a method for administering a pharmaceutical composition including the composition of the present invention to a subject in need thereof is provided. In some exemplary embodiment, the composition of the present invention may be administered to a human.

The compound of the present invention may be used in the form of a pharmaceutically acceptable salt, and as the salt, an acid addition salt formed by a pharmaceutically acceptable free acid is useful. The expression pharmaceutically acceptable salt refers to any organic or inorganic addition salt of a base compound of

Chemical Formula 1 where side effects resulting from the salt do not degrade the beneficial efficacy of the base compound of Chemical Formula 1 at a concentration that is relatively non-toxic and harmless to the patients and has an effective action. These salts may use an inorganic acid and an organic acid as a free acid, as the inorganic acid, it is possible to use hydrochloric acid, bromic acid, nitric acid, sulfuric acid, perchloric acid, phosphoric acid, and the like, and as the organic acid, it is possible to use citric acid, acetic acid, lactic acid, maleic acid, fumaric acid, gluconic acid, methanesulfonic acid, glycolic acid, succinic acid, tartaric acid, galacturonic acid, embonic acid, glutamic acid, aspartic acid, oxalic acid, (D) or (L) malic acid, maleic acid, methanesulfonic acid, ethanesulfonic acid, 4-toluenesulfonic acid, salicylic acid, citric acid, benzoic acid, malonic acid, and the like. Further, these salts include alkali metal salts (sodium salts, potassium salts, and the like), alkaline earth metal salts (calcium salts, magnesium salts, and the like), and the like. For example, as an acid addition salt, acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulfate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salts, and the like may be included.

The pyrrolopyrimidinone carboxamide compound represented by Chemical Formula 1 of the present invention, a stereoisomer thereof, or a pharmaceutically acceptable salt thereof includes not only pharmaceutically acceptable salts, but also all salts, isomers, hydrates and solvates that can be prepared by typical methods.

The addition salt according to the present invention may be prepared by a typical method, and may be prepared, for example, by dissolving the compound of Compound Formula 1 in a water-miscible organic solvent, for example, acetone, methanol, ethanol, or acetonitrile, or the like, adding an excessive amount of an organic acid thereto or adding an aqueous acid solution of an inorganic acid thereto, followed by precipitation or crystallization. Subsequently, the acid addition salt may be prepared by evaporating the solvent or excess acid from this mixture, and then drying the mixture or suction-filtering a precipitated salt.

When the composition of the present invention is used as a drug, a pharmaceutical composition containing the pyrrolopyrimidinone carboxamide derivative of Chemical Formula 1 or a pharmaceutically acceptable salt thereof as an active ingredient may be formulated and administered in various oral or parenteral dosage forms as described below at the time of clinical administration, but is not limited thereto.

Examples of a formulation for oral administration include a tablet, a pill, a hard/soft capsule, a solution, a suspension, an emulsion, a syrup, a granule, an elixir, and the like, and these formulations contain a diluent (for example: lactose, dextrose, sucrose, mannitol, sorbitol, cellulose and/or glycine) and a lubricant (for example: silica, talc, stearic acid, and a magnesium or calcium salt thereof, and/or polyethylene glycol) in addition to an active ingredient. The tablet may also contain a binder such as magnesium aluminum silicate, starch paste, gelatin, methylcellulose, sodium carboxymethylcellulose, and/or polyvinylpyrrolidine, and may contain a disintegrant such as starch, agar, alginic acid, or a sodium salt thereof or a boiling mixture, and/or an absorbent, a colorant, a flavoring agent, and a sweetening agent in some cases.

A pharmaceutical composition including the pyrrolopyrimidinone carboxamide derivative of Chemical Formula 1 as an active ingredient may be parenterally administered, and the parenteral administration is performed using an injection method such as subcutaneous injection, intravenous injection, intramuscular injection, or intrathoracic injection.

In this case, in order to formulate the pharmaceutical composition into a formulation for parenteral administration, the pharmaceutical composition may be prepared into a solution or suspension by mixing the pyrrolopyrimidinone carboxamide compound of Chemical Formula 1, the stereoisomer thereof, or the pharmaceutically acceptable salt thereof with a stabilizer or buffer in water and the solution or suspension may be prepared in an ampoule or vial unit form for administration. The composition may be sterilized and/or contain an adjuvant such as a preservative, stabilizer, hydrating agent, or an emulsion-promoting agent, a salt and/or buffer for adjusting osmotic pressure, and other therapeutically useful materials, and the composition may be formulated using a typical method such as a mixing, granulating, or coating method.

Further, the dose of the compound of the present invention administered to the human body may vary depending on the age, body weight, gender, administration form, health status, and level of disease of a patient, and is generally 0.1 to 1,000 mg/day, preferably 1 to 500 mg, based on an adult patient having a body weight of 70 kg, and the composition may be administered once or in divided doses several times a day at predetermined time intervals depending on the judgment of a doctor or a pharmacist.

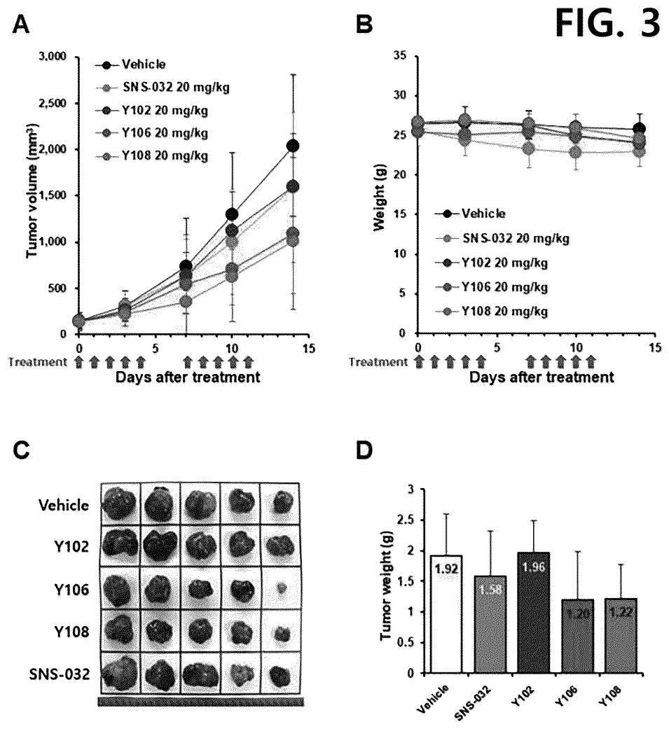

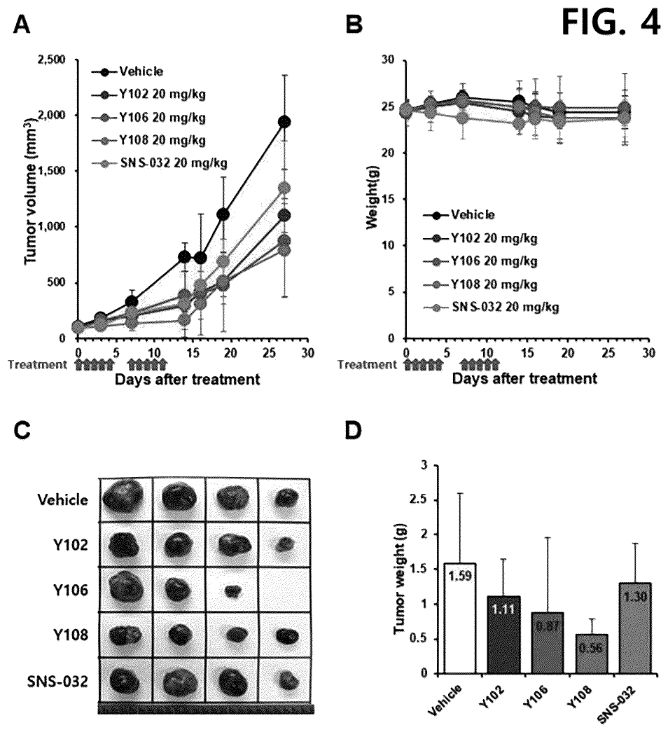

In a specific exemplary embodiment of the present invention, it was confirmed that the compound of the present invention inhibited the growth of and reduced the weight of Huh-7 cells, a hepatocellular carcinoma cell line (Example 3 and FIG. 3), and also reduced the growth and weight of cancer cells in Hep3B cells, another hepatocellular carcinoma cell line (Example 4 and FIG. 4).

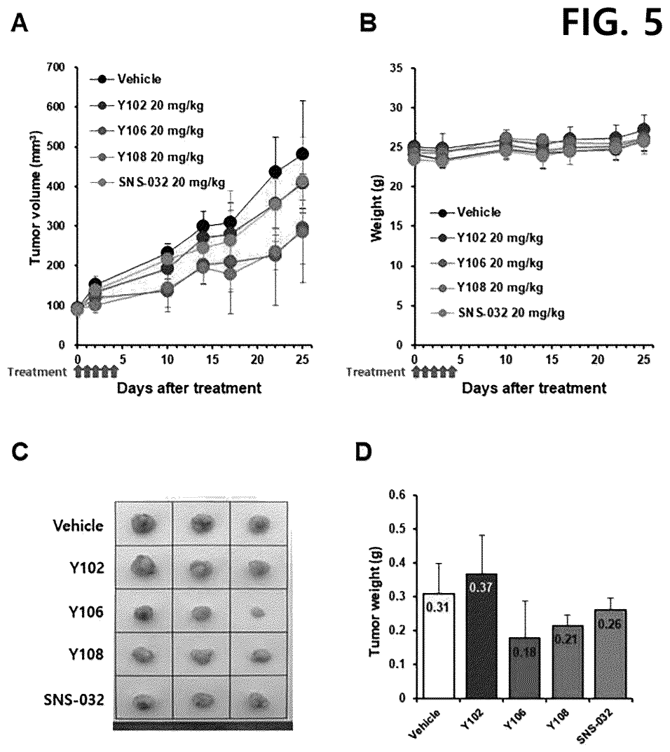

In addition, as a result of evaluating the efficacy of the compound of the present invention in a mouse model transplanted with A549 cells, a non-small cell lung cancer cell line, it was confirmed that the growth and weight of cancer cells were reduced (Example 5 and FIG. 5).

In an exemplary embodiment, the cancer may be liver cancer or lung cancer. “Lung cancer” means a malignant tumor originating in the lungs, and is roughly divided into small cell lung cancer and non-small cell lung cancer according to the tissue morphology thereof. In an exemplary embodiment, preferably, the lung cancer may be non-small cell lung cancer.

In an exemplary embodiment, the composition may be characterized as having a CDK inhibitory action. In an exemplary embodiment, the CDK may be any one selected from the group consisting of Cdk1, Cdk2, Cdk7, and Cdk9.

The present invention provides a CDK inhibitor including the pyrrolopyrimidinone carboxamide compound represented by Chemical Formula 1.

The present invention provides a method for inhibiting CDK in a specimen or cell, the method including administering a pharmaceutically effective amount of the pyrrolopyrimidinone carboxamide compound represented by Chemical Formula 1 to the specimen.

The present invention provides a method for preventing or treating cancer in a specimen, the method including administering a pharmaceutically effective amount of the pyrrolopyrimidinone carboxamide compound represented by Chemical Formula 1 to the specimen.

The present invention provides a use of the pyrrolopyrimidinone carboxamide compound represented by Chemical Formula 1 for preventing or treating cancer or tumors.

Hereinafter, the present invention will be described in more detail through examples. These examples are only for exemplifying the present invention, and it should be obvious to a person with ordinary skill in the art that the scope of the present invention is not to be interpreted as being limited by these Examples.

### EXAMPLE 1

### Synthesis of Compound

¹H and ¹³C NMR-spectra were measured with an Agilent 400-MR DD2 magnetic resonance system (400 MHZ) and a Varian/Oxford As-500 (500 MHZ) spectrophotometer. Chemical shifts were measured in parts per million (δ values) from tetramethylsilane as an internal standard at a probe temperature in chloroform-d or acetone-D6 for neutral compounds. Coupling constants are provided in Hz, with the following spectral pattern designations. s, singlet; d, doublet; t, triplet; q, quartet; quint, quintet; m, multiplet; br, broad; app, apparent. Reactions that needed conditions were carried out in flame-dried glassware under a positive pressure of dry N₂ using standard Schlenk line techniques. Evaporation of solvents was performed under reduced pressure using a rotary evaporator. TLC was performed using silica gel 60F254 coated on an aluminum sheet (E. Merck, Art. 5554). Column chromatography was performed on a silica gel (Merck. 7734 or 9385 Kiesel gel 60), and an eluent was mentioned in each procedure. Elemental analyses were performed on a Thermo Scientific model Flash 2000 instrument (Waltham, MA, USA), and results are within ±0.4% of theoretical values. High resolution mass spectra (HRMS) were recorded on a ThermoFinnigan LCQ™ Classic, Quadrupole Ion-Trap Mass Spectrometer. HPLC analyses were carried out on an Agilent HP1100 system (Santa Clara, CA, USA) composed of an auto sampler, a quaternary pump, a photodiode array detector (DAD), and HP Chemstation software. Separation was carried out on a Poroshell 120 ECC18 column 4.6×50 mm (2.7 um particle size). 0.1% TFA in water (A) and acetonitrile (B) were used as a mobile phase at a flow rate of 1 mL/min at 20° C. Method: 100% A and 0% B (0 min), 50% A and 50% B (5 min), 5% A and 95% B (15 min), 5% A and 95% B (22 min), 100% A and 0% B (23 min), 100% A and 0% B (25 min). Details are given below for one example of each reaction type. All materials were obtained from commercial suppliers and used without further purification unless otherwise specified.

### <1-1>Preparation of (2S,3S,4S,5S)-5-(4-amino-6-bromo-5-carbamoyl-1H-pyrrolo[2,3-d]pyrimidin-1-yl)-4-hydroxy-2-((isobutyryloxy)methyl)tetrahydrofuran-3-yl isobutyrate (BMK-Y105, 2)

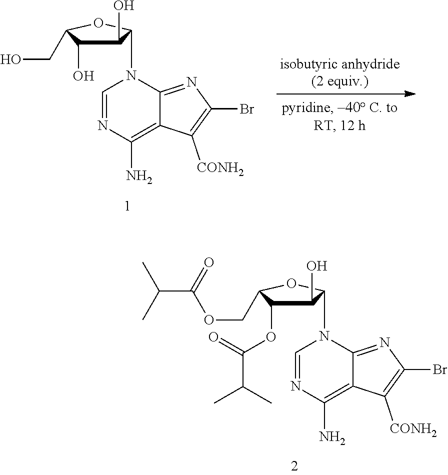

Compound 1 (387 mg, 1.00 mmol) was dissolved in pyridine (5 mL). To this solution, isobutyric anhydride (350 μL, 2.1 mmol) was slowly added at-40° C. over 30 minutes using a syringe pump. The reaction mixture was stirred at room temperature for 12 hours. The mixture was poured into water (20 mL) and extracted with EtOAc (3×20 mL). The combined EtOAc extract was washed with a saturated NaHCO₃ solution and a brine solution, dried (MgSO₄), and concentrated. A desired product was isolated by flash chromatography to obtain double-protected compound 2 (158 mg, 30%).

¹H NMR (500 MHz, chloroform-d) δ 10.43 (d, J=4.9 Hz, 1H), 8.30 (d, J=11.3 Hz, 1H), 8.04 (s, 1H), 7.14 (s, 1H), 5. (s, 1H), 5.65 (s, 1H), 5.45 (s, 1H), 4.70 (dd, J=11.9, 3.4 Hz, 1H), 4.47-4.29 (m, 3H), 2.61 (dp, J=32.8, 7.1 Hz, 2H), 1.28-1.08 (m, 12H). ¹³C NMR (125 MHz, chloroform) δ 177.14, 176.98, 166.11, 157.76, 144.15, 143.23, 124.91, 108.21, 104.91, 97.33, 83.88, 81.59, 75.01, 62.60, 33.82, 33.68, 18.9.9.93.93, 18.93, 18.93. HRMS (ESI) m/z: Anal. calcd. [M+H]+ C₂₀H₂₇BrN₅O₇: 528.1094, found 528.1079. HPLC purity: 99.9%.

### <1-2>Preparation of (2S,3S,4S,5S)-5-(4-amino-6-bromo-5-carbamoyl-1H-pyrrolo[2,3-d]pyrimidin-1-yl)-2-(((2,2-dimethylbutanoyl)oxy)methyl)-4-hydroxytetrahydrofuran-3-yl 2,2-dimethylbutanoate (BMK-Y106, 3)

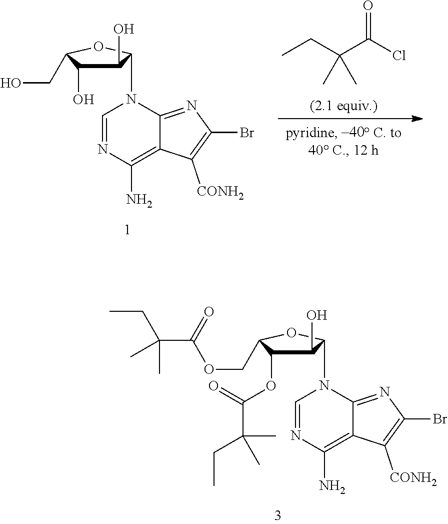

Compound 1 (387 mg, 1.00 mmol) was dissolved in pyridine (5 mL). To this solution, 2,2-dimethylbutyryl chloride (288 μL, 2.1 mmol) was slowly added at −40° C. over 30 minutes using a syringe pump. The reaction mixture was stirred at 40° C. for 12 hours. The mixture was poured into water (20 mL) and extracted with EtOAc (3×20 mL). The combined EtOAc extract was washed with a saturated NaHCO₃ solution and a brine solution, dried (MgSO₄), and concentrated. A desired product was isolated by flash chromatography to obtain double-protected compound 3 (129 mg, 22%).

¹H NMR (500 MHz, chloroform-d) δ 10.61-10.49 (m, 1H), 8.17 (s, 1H), 7.98 (s, 1H), 7.18 (s, 1H), 5.76 (s, 1H), 5 s, 1H), 5.39 (d, J=2.0 Hz, 1H), 4.69 (dd, J=12.2, 3.6 Hz, 1H), 4.48-4.33 (m, 2H), 4.25 (dt, J=7.2, 3.5, 1H), 1.59 (dq, J=12.6, 7.4 Hz, 4H), 1.22-1.13 (m, 13H), 0.85 (dt, J=14.8, 7.4 Hz, 6H). ₁₃C NMR (125 MHZ, chloroform) δ 178.90, 178.33, 166.42, 158.25, 144.63, 143.67, 125.29, 108.66, 105.33, 97.74, 84.49, 82.12, 75.47, 63.00, 43.12, 33.72, 24, 24.4, 24.4, 24.0, 24.4, 24.4, 24.4, 24.4, 9.70. HRMS (ESI) m/z: Anal. calcd. [M+H]+ C₂₄H₃₅BrN₅O₇: 584.1720, found 584.1700. HPLC purity: 99.9%.

### <1-3>Preparation of ((2S,3S,4S,5S)-5-(4-amino-6-bromo-5-carbamoyl-1H-pyrrolo[2,3-d]pyrimidin-1-yl)-3,4-dihydroxytetrahydrofuran-2-yl)methyl 2,2-dimethylbutanoate (BMK-Y107, 4)

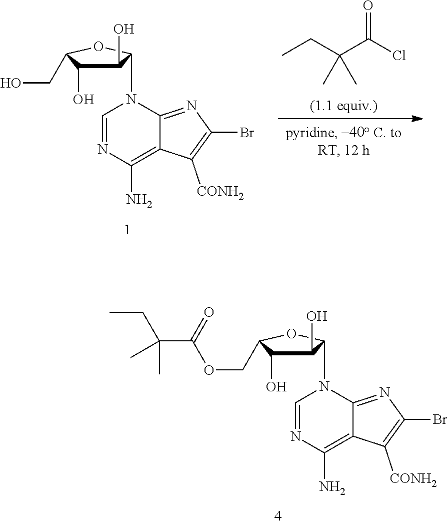

Compound 1 (387 mg, 1.00 mmol) was dissolved in pyridine (5 mL). To this solution, 2,2-dimethylbutyryl chloride (151 μL, 1.1. mmol) was slowly added at −40° C. over 30 minutes using a syringe pump. The reaction mixture was stirred at room temperature for 12 hours. The mixture was poured into water (20 mL) and extracted with EtOAc (3×20 mL). The combined EtOAc extract was washed with a saturated NaHCO₃ solution and a brine solution, dried (MgSO₄), and concentrated. A desired product was isolated by flash chromatography to obtain mono-protected compound 4 (126 mg, 26%).

¹H NMR (400 MHZ, acetone-d6) δ 10.56 (s, 1H), 8.48 (s, 1H), 7.50 (s, 1H), 7.21 (s, 1H), 6.86 (s, 1H), 6.22 (s, 1H) 1H), 5.99 (d, J=1.9 Hz, 1H), 5.42-5.29 (m, 1H), 4.68 (s, 1H), 4.59-4.38 (m, 3H), 4.22 (s, 1H), 1.57 (q, J=7.5 Hz, 2H), 1.14 (d, J=2.5 Hz, 6H), 0.83 (t, J=7.5 Hz, 3H). ¹³C NMR (125 MHz, acetone-d6) δ 206.15, 144.36, 97.88, 82.48, 81.91, 77.77, 63.34, 33.97, 24.98, 24.97, 9. Anal. calcd. [M+H]+ C₁₈H₂₅BrN₅O₆: 486.0988, found 486.0979. HPLC purity: 99.9%.

### <1-4>Preparation of (2S,3S,4S,5S)-5-(4-amino-6-bromo-5-carbamoyl-1H-pyrrolo[2,3-d] pyrimidin-1-yl)-4-hydroxy-2-((pivaloyloxy)methyl)tetrahydrofuran-3-yl pivalate (BMK-Y108, 5)

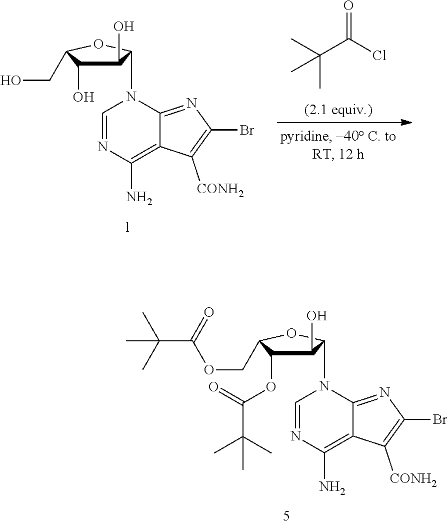

Compound 1 (387 mg, 1.00 mmol) was dissolved in pyridine (5 mL). To this solution, trimethylacetyl chloride (258 μL, 2.1.mmol) was slowly added at −40° C. over 30 minutes using a syringe pump. The reaction mixture was stirred at room temperature for 12 hours. The mixture was poured into water (20 mL) and extracted with EtOAc (3×20 mL). The combined EtOAc extract was washed with a saturated NaHCO₃ solution and a brine solution, dried (MgSO₄), and concentrated. A desired product was isolated by flash chromatography to obtain double-protected compound 5 (145 mg, 26%).

¹H NMR (500 MHz, chloroform-d) δ 10.48 (d, J=5.0 Hz, 1H), 8.27 (d, J=11.3 Hz, 1H), 8.01 (s, 1H), 7.16 (s, 2H), 5.27 (s, 1H), 5.58 (s, 1H), 5.40 (s, 1H), 4.69 (dd, J=12.2, 3.3 Hz, 1H), 4.45-4.39 (m, 1H), 4.35 (dd, J=12.2, 7.6 Hz, 1H), 4.27 (dt, J=7.3, 3.4 Hz, 1H), 1.22 (d, J=14.3 Hz, 18H). ¹³C NMR (125 MHZ, chloroform) δ 178.83, 178.63, 166.38, 157.96, 144.36, 143.53, 125.25, 108.41, 105.20, 97.56, 84.00, 81.85, 75.31, 62.98, 38.97, 38.95, 27.38, 38.97. Anal. calcd. [M+H]+ C₂₂H₃₁BrN₅O₇: 556.1407, found 556.1400. HPLC purity: 99.9%.

### <1-5>Preparation of ((2S,3S,4S,5S)-5-(4-amino-6-bromo-5-carbamoyl-1H-pyrrolo[2,3-d]pyrimidin-1-yl)-3,4-dihydroxytetrahydrofuran-2-yl)methyl(3S,5S,7S)-adamantane-1-carboxylate (BMK-Y109,6)

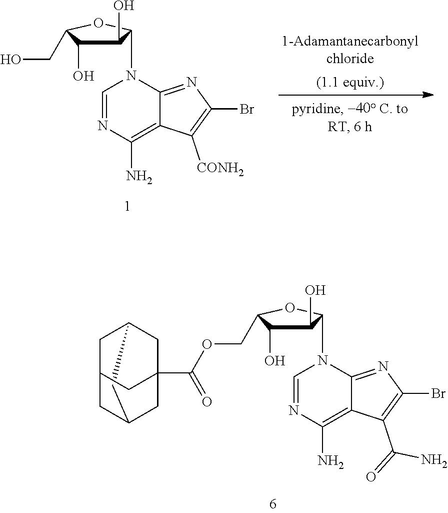

Compound 1 (387 mg, 1.00 mmol) was dissolved in pyridine (5 mL). To this solution, 1-adamantanecarbonyl chloride (182 μL, 1.1.mmol) was slowly added at −40° C. over 30 minutes using a syringe pump. The reaction mixture was stirred at room temperature for 6 hours. The mixture was poured into water (20 mL), and the combined EtOAc extract was washed with a saturated NaHCO₃ solution and a brine solution, dried (MgSO₄), and concentrated. A desired product was isolated by flash chromatography to obtain mono-protected compound 6 (138 mg, 25%).

¹H NMR (400 MHz, acetone-d6) δ 10.54 (s, 1H), 8.48 (s, 1H), 7.51 (s, 1H), 7.21 (s, 1H), 6.88 (s, 1H), 6.17 (s, 1H) 1H), 5.99 (s, 1H), 5.47 (s, 1H), 4.68 (s, 1H), 4.47 (dt, J=18.7, 6.6 Hz, 3H), 4.25 (s, 1H), 1.96 (s, 3H)), 1.87 (s, 6H), 1.78-1.64 (m, 6H). ¹³C NMR (125 MHz, acetone-d6) δ 206.20, 144.25, 97.77, 82.46, 81.86, 81.84, 77.62, 63.19, 39.51, 37.10, 28 Anal. calcd. [M+H]+ C₂₃H₂₉BrN₅O₆: 550.1301, found 550.1296. HPLC purity: 99.9%.

### EXAMPLE 2

### Plasma Metabolic Stability Assay

### Solution Preparation

DMSO was used as a diluent to prepare stock solutions of a test substance and a positive control at a concentration of 10 mM. The positive control stock solution was then diluted with 70% acetonitrile to a working concentration of 0.2 mM, and then the test substance stock solution was diluted with 70% acetonitrile to a working concentration of 1 mM. A quench reagent consisted of acetonitrile containing tolbutamide and propranolol (provided as an internal standard).

### Analytical Procedure

Positive control and test substance working solutions were spiked into plasma at a concentration of 1 μM and 5 μM, respectively. A 0-min sample was prepared by adding an 80 μL aliquot of each incubation mixture to 320 μL of the quench reagent to precipitate proteins. The mixture was incubated in a water bath at 37° C. with gentle shaking. Aliquots of 80 μL of each mixture were removed and placed in a clean 96-well plate containing 320 μL of the quench reagent to precipitate proteins at 15, 30, 45, and 60 min and centrifuged (5000×g, 15 minutes). 100 μL of the supernatant was placed in a 96-well assay plate to which 200 μL of ultrapure water had been added in advance, and analyzed by LC-MS/MS.

The results are described in the following [Table 1].

TABLE 1

T1/2(min)

| | Treatment | Mouse | Human |
|---|---|---|---|
| Compound | Ibulocydine | 2.85 ± 0.02 | 4.57 ± 0.09 |
| | Y105 | 2.67 ± 0.20 | 20.53 ± 0.34 |
| | Y106 | 7.10 ± 0.07 | >720 |
| | Y107 | 6.91 ± 0.11 | 26.55 ± 4.33 |
| | Y108 | 7.25 ± 0.01 | 700.29 ± 42.54 |
| | Y109 | 7.77 ± 0.13 | 280.32 ± 3.06 |
| Reference | Eucatropin | 32.57 ± 4.81 | 16.73 ± 0.38 |

Compounds BMK-Y106 and BMK-Y108 were found to have the highest stability in each group. Therefore, it was found that the number of protecting groups and the steric effect significantly affected the half-life of the compound.

### EXAMPLE 3

### Evaluation of Anticancer Efficacy of Y106 and Y108 in Mouse Model Transplanted With Huh-7 Cells, Hepatocellular Carcinoma Cell Line

To evaluate in vivo efficacy, 1×10⁷ Huh-7 cells were transplanted into the right calves of nude mice to form cancer tissue, and then the anticancer efficacy of the new compositions Y106 and Y108 was compared with that of an existing CDK inhibitor Y102 or SNS-032.

The dose was the same for all mice (20 mg/kg), the vehicle, Y102, Y106, and Y108 were administered orally, and only SNS-032 was administered intraperitoneally. These were administered five times a week for two weeks, the size was measured twice a week using calipers to measure the changes in cancer growth and the mouse body weight for 14 days, and on day 14, the cancer tissue was removed to confirm the size and weight thereof.

As a result, as shown in FIG. 3, it was confirmed that the groups administered Y106 and Y108 had significantly inhibited cancer growth compared to the groups administered Y102 and SNS-032 (A of FIG. 3), and in this case, the size of the cancer was calculated by the following equation.

$$Cancer size ( Tumor volume ) = [ length \times {width}^{2} ] \times 0.5$$

In addition, it was confirmed that the body weight of the mouse was slightly reduced only in the case of SNS-032 (B of FIG. 3), and when the cancer tissues of all the mice were also removed (C of FIG. 3) to measure the weight at the end of the experiment, the groups administered Y106 and Y108 had the least amount of cancer tissue (D of FIG. 3).

### EXAMPLE 4

Evaluation of anticancer efficacy of Y106 and Y108 in mouse model transplanted with Hep3B cells, hepatocellular carcinoma cell line

To evaluate in vivo efficacy using Hep3B cells, another hepatocellular carcinoma cell line, 3×10⁶ Hep3B cells were transplanted into the right calves of nude mice to form cancer tissue, and then the anticancer efficacy of the new compositions Y106 and Y108 was compared with that of an existing CDK inhibitor Y102 or SNS-032.

The dosage, administration schedule, and administration route were applied in the same manner as in the method performed in the mouse model transplanted with Huh-7 cells in Example 3. After administration of the new compositions and the existing CDK inhibitors, changes in cancer growth and mouse body weight were measured for 27 days, and then on day 27, the cancer tissues were removed to confirm the size and weight thereof.

As a result, as shown in FIG. 4, similar to the results in the mouse model transplanted with Huh-7 cells, it was confirmed that the groups administered Y106 and Y108 inhibited cancer growth more effectively than the groups administered Y102 and SNS-032 (A of FIG. 4), and there was no significant difference in the changes in mouse body weight (B of FIG. 4). It was confirmed that when the cancer tissues of all the mice were also removed (C of FIG. 4) to measure the weight at the end of the experiment, the groups administered Y106 and Y108 had the least amount of cancer tissue (D of FIG. 4).

### EXAMPLE 5

### Evaluation of Anticancer Efficacy of Y106 and Y108 in Mouse Model transplanted with A549 cells, non-small cell lung cancer cell line

To evaluate in vivo efficacy in a mouse model transplanted with A549 cells, a non-small cell lung cancer cell line, 1×10⁶ A549 cells were transplanted into the right calves of nude mice to form cancer tissue, and then the anticancer efficacy of the new compositions Y106 and Y108 was compared with that of an existing CDK inhibitor Y102 or SNS-032.

The dose was the same for all mice (20 mg/kg), the vehicle, Y102, Y106, and Y108 were administered orally, and only SNS-032 was administered intraperitoneally. These were administered for five days, the size was measured twice a week using calipers to measure the changes in cancer growth and the mouse body weight for 25 days, and on day 25, the cancer tissue was removed to confirm the size and weight thereof.

As a result, as shown in FIG. 5, it was confirmed that the groups administered Y106 and Y108 inhibited cancer growth more clearly than the groups administered Y102 and SNS-032 (A of FIG. 5), and there was no significant difference in the changes in mouse body weight (B of FIG. 5). It was confirmed that when the cancer tissues of all the mice were also removed (C of FIG. 5) to measure the cancer weight at the end of the experiment, the groups administered Y106 and Y108 had the least amount of cancer tissue (D of FIG. 5).

### EXAMPLE 6

### Cell Colony Forming Assay

To examine the anticancer efficacy of Y106 and Y108 in vitro, a cell colony formation assay was performed in hepatocellular carcinoma cell lines Huh-7 and Hep3B cells.

500 cells were seeded into 6-well plates, and then treated with Y102, Y106,Y108, or SNS-032 at 1 μM and 3 μM for 12 hours, the medium was then replaced with fresh medium, and after 7 to 14 days, the cells were stained with 20% methanol+0.5% Crystal Violet for colony staining, and the number of cells was counted for quantitative analysis.

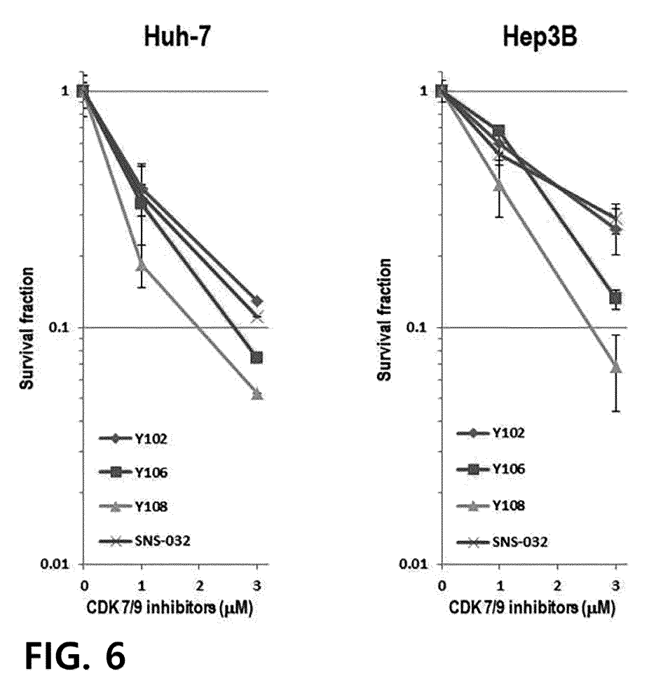

As a result of the assay, it could be seen that colony formation was remarkably reduced in both cells when they were treated with Y106 and Y108 compared to when they were treated with Y102 or SNS-032, and the results are shown in FIG. 6.

In summary, the present invention relates to a composition for treating hepatocellular carcinoma and non-small cell lung cancer, which contains a new pyrrolopyrimidinone carboxamide derivative that inhibits CDK as an active ingredient.

As a result of comparing the anticancer efficacy of the newly synthesized Y106 and Y108 in a mouse model transplanted with hepatocellular carcinoma cell lines Huh-7 and Hep3B cells with that of the existing CDK inhibitors Y102 and SNS-032, it was confirmed that Y106 and Y108 had excellent anticancer efficacy. In addition, it was confirmed that the anticancer efficacy of Y106 and Y108 is also better than that of Y102 or SNS-032 in a mouse model transplanted with A549 cells, a non-small cell lung cancer cell line. Furthermore, a cell colony forming assay also demonstrated that Y106 and Y108 remarkably reduced the cell colony formation compared to Y102 and SNS-032. Therefore, the compound according to the present invention can be usefully used as an active ingredient in a pharmaceutical composition for treating hepatocellular carcinoma or non-small cell lung cancer.

1. A pyrrolopyrimidinone carboxamide compound represented by the following Chemical Formula 1, a stereoisomer thereof, or a pharmaceutically acceptable salt thereof: 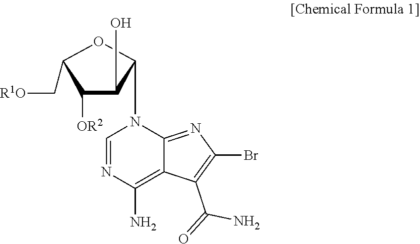
wherein R¹ is R³C(═O),
R³ is any one selected from the group consisting of a straight or branched C₃-C₁₀ alkyl, a C₃-C₁₀ cycloalkyl, and a C₆-C₁₀ aryl,
R² is hydrogen or R⁴C(═O), and R⁴ is any one selected from the group consisting of straight or branched C₃-C₆ alkyls, and
when R³ is propyl or butyl, R² is not hydrogen.

2. The pyrrolopyrimidinone carboxamide compound, the stereoisomer thereof, or the pharmaceutically acceptable salt thereof of claim 1, wherein R³ is any one selected from the group consisting of isopropyl, butyl, pentyl, and adamantyl, and
R⁴ is any one selected from the group consisting of isopropyl, butyl, and pentyl.

3. The pyrrolopyrimidinone carboxamide compound, the stereoisomer thereof, or the pharmaceutically acceptable salt thereof of claim 1, wherein the compound represented by Chemical Formula 1 is selected from the group consisting of
(1) (2S,3S,4S,5S)-5-(4-amino-6-bromo-5-carbamoyl-1H-pyrrolo[2,3-d]pyrimidin-1-yl)-4-hydroxy-2-((isobutyryloxy)methyl)tetrahydrofuran-3-yl isobutyrate;
(2) (2S,3S,4S,5S)-5-(4-amino-6-bromo-5-carbamoyl-1H-pyrrolo[2,3-d]pyrimidin-1-yl)-2-(((2,2-dimethylbutanoyl)oxy)methyl)-4-hydroxytetrahydrofuran-3-yl 2,2-dimethylbutanoate;
(3) ((2S,3S,4S,5S)-5-(4-amino-6-bromo-5-carbamoyl-1H-pyrrolo[2,3-d]pyrimidin-1-yl)-3,4-dihydroxytetrahydrofuran-2-yl)methyl 2,2-dimethylbutanoate;
(4) (2S,3S,4S,5S)-5-(4-amino-6-bromo-5-carbamoyl-1H-pyrrolo[2,3-d]pyrimidin-1-yl)-4-hydroxy-2-((pivaloyloxy)methyl)tetrahydrofuran-3-yl pivalate; and
(5) ((2S,3S,4S,5S)-5-(4-amino-6-bromo-5-carbamoyl-1H-pyrrolo[2,3-d]pyrimidin-1-yl)-3,4-dihydroxytetrahydrofuran-2-yl)methyl(3S,5S,7S)-adamantane-1-carboxylate.

4. A pharmaceutical composition for preventing or treating cancer, comprising the compound, the stereoisomer thereof, or the pharmaceutically acceptable salt thereof of claim 1 as an active ingredient.

5. The pharmaceutical composition of claim 4, wherein the cancer is liver cancer or lung cancer.

6. The pharmaceutical composition of claim 5, wherein the lung cancer is non-small cell lung cancer.

7. The pharmaceutical composition of claim 4, wherein the composition has a CDK inhibitory action.

8. The pharmaceutical composition of claim 7, wherein the CDK comprises any one selected from the group consisting of Cdk1, Cdk2, Cdk7, and Cdk9.

9. (canceled)

10. (canceled)

11. A method for preventing or treating cancer in a specimen, the method comprising administering a pharmaceutically effective amount of the compound of claim 1 to the specimen.

12. (canceled)
